(19) United States
(12) Patent Application Publication
(10) Pub. No.: US 2006/0246416 A1
(43) Pub. Date: Nov. 2, 2006

(54) On-chip bioassay method and kit

(76) Inventors: Hirofumi Tani, Hokkaido (JP); Tamio Kamidate, Hokkaido (JP); Koji Maehana, Hokkaido (JP)

Correspondence Address: Kenneth H. Sommenfeld;Morgan & Financial, 3 World Financial Center, New York, NY, 10281-2101, US

(21) Appl. No.: 10/566,861

(22) PCT Filed: Feb. 4, 2004
(86) PCT No.: PCT/JP04/01128

§ 371 Date: Jun. 30, 2006

(30) Foreign Application Priority Data
Jul. 31, 2003 (JP) 2003-284208

(51) Int. Cl. C12Q 1/00; C12Q 1/68; C12M 1/34

(52) U.S. Cl. 435/4; 435/6; 435/287.2

(57) ABSTRACT

The present invention provides an on-chip bioassay system which reduces preoperations including culture, and is highly versatile and capable of conducting high throughput analyses. A microfluidic chip for introducing a cell is fixed on the undersurface of a microporous chip composed of a substrate through which a plurality of micropores arranged like a lattice penetrate, and thus forming a plurality of microchannels between the microporous chip and the microfluidic chip for introducing a cell; a suspended cell is poured into a micropore through the channel; then a microfluidic chip for introducing a test substance is fixed on the upper surface of the microporous chip such that its plurality of microchannels for introducing a test substance are placed in a direction perpendicular to the channels for introducing a cell, and thus forming a plurality of microchannels between the microporous chip and the microfluidic chip for introducing a test substance; a test substance is poured through the channel and brought into contact with the cell in a micropore of the microporous chip; a level of effect of the test substance on the cell is detected in situ after a predetermined time, or at a predetermined time interval.

### TECHNICAL FIELD

The present invention relates to an on-chip bioassay method and an on-chip bioassay kit used for the method.

### BACKGROUND ART

With the development of fine processing technology in recent semiconductor industry, analytical instruments using microchips wherein factors necessary for chemical analyses such as microchannels, microreactors, and microelectrodes for detection are integrated on a substrate made of silicon or glass, have come into use. Microchip-based electrophoresis apparatuses for DNAs or proteins are already developed and commercially available. By analytical devices on the basis of microfluidic chips thus described (microanalytical system, μ-Total Analysis System, μ-TAS), integration, high throughput, resource saving, space saving and low emission of chemical analysis experiments can be achieved. Currently, separation chips for the above-mentioned electrophoresis or chromatography mainly focused on biochemical analyses, assay chips for immunoassays or enzymatic analyses, chips for synthetic reaction used for polymerase chain reaction (PCR), etc., are actively developed on a global basis. As these chips are easily portable, it is expected that environmental analyses can be conducted on the spot of sampling, and that highly accurate clinical trials can be conducted at bed side.

Further, microarray can be exemplified also as a microchip-based analytical device. In this device, DNAs, proteins, etc., are high-densely arrayed on solid substrates and immobilized as probe molecules, and mRNA or protein expression, and interactions between biomolecules can be analyzed by monitoring the binding of DNAs, RNAs, proteins, substrate molecules in samples to the probe molecules. In these arrayed-type microchips, a number of probes are immobilized on one substrate, however, samples capable of reacting with such probes on the substrates are single specimens, and it is not intended to monitor the combination with many specimens simultaneously.

In conventional microchip-based analytical systems, quantification of target molecules and evaluation of interactions are conducted by monitoring the interactions between probe molecules and target molecules, and subsequent reactions. When evaluating the properties of environmental pollutants and novel chemical substances, however, it is necessary to monitor not only information regarding concentrations and interactions with biomolecules, but also biological responses caused by them. To achieve such purpose, bioassays, with the use of living bodies, organs, particular cells, or cells whose function is transformed by genetic recombination, need to be conducted.

On the other hand, a substrate for high density cell array used for providing a culture substrate, a cell array, an automatic injector device for chemical substances, an assay system, which are necessary for easy assays of chemical substances such as various kinds of pharmaceuticals and poisons, wherein the substrate for high density cell array has a surface in which areas coated with a cell-adhesive polymer are discontinuously, finely and regularly arranged, surrounded with an area coated with a cell-nonadhesive hydrophilic polymer, and further surrounded continuously with an area coated with a cell-nonadhesive strongly hydrophobic material (Japanese Laid-Open Patent Application No. 2003-33177), a method of preparing a cell array that is particularly useful for conducting comparative cell-based analyses, which permits high throughput screening for candidate modulators of a signal transduction pathway, and comprises the following steps: (a) providing an array of tubes, each tube having at least one lumen and a population of cells that is contained within said lumen; (b) cross-sectioning the array of tubes to yield a plurality of transverse tube segments; and (c) immobilizing the plurality of tube segments on a solid support (Published Japanese translation of PCT international publication No. 2003-516747), and other such techniques regarding cell arrays and cell chips (for example, Japanese Laid-Open Patent Application No. 2002-297922, and Published Japanese translation of PCT international publication Nos. 2002-543429 and 2002-523781) have been proposed.

In addition, a method for assaying test samples for a biological or biochemical activity comprising the steps of: introducing multiple test samples onto a matrix capable of simultaneously assaying large numbers of chemical entities for a wide range of biological or biochemical activity; b) using at least one matrix to introduce one or more assay component to the assay wherein the matrix may or may not be the porous assay matrix; and c) performing the additional step of i) washing any matrix used in the assay to remove an excess amount of test sample, assay component or a combination thereof; or ii) contacting any matrix used in the assay with an additional reagent in bulk solution or as a liquid (Published Japanese translation of PCT international publication No. 2001-526390) has been proposed.

When performing various analyses and tests using cells and bacteria, usually it is necessary to perform a series of operations such as cell culture, addition and detection of samples, with the use of test tubes, microtiter plates, etc. These operations involving cell handling need special facilities and skills required for aseptic manipulation, therefore, these operations have a problem of low versatility. Further, as cell culture conducted just before the tests usually takes long time, it causes a problem particularly when many samples are used, and when plural kinds of cells are used for extracting a lot of information.

In order to solve these problems, a highly versatile testing system that does not make operators aware of cell handling, and reduces preoperations including culture, is required. In fields of current chemical and biochemical analyses, various test strips, test kits, sensors, etc., which meet these needs are developed and commercially available, however, simplified test methods thus described have not come into practical use in the field of bioassay. Further, for high throughput analyses, systems that can achieve combinations of many samples and many probes (for instance, cells in case of bioassay) simultaneously are also necessary. Recently, there are attempts to monitor the effect of chemical substances and bioactive substances on cells by using microchips, however, these attempts have not been practically applied so far because versatile cell immobilization techniques and signal detection methods are not sufficiently established.

The object of the present invention is to provide a bioassay system that does not make operators aware of cell handling and reduces preoperations including culture, and is highly versatile and capable of conducting high throughput analyses.

The present inventors have conducted keen study to attain the above-mentioned object, and contrived a microchip wherein plural kinds of cells are immobilized in plural micropores on a substrate. By immobilizing cells which have been cultured to the extent that they can be used for examinations, operators can use the cells for examinations immediately without preoperations, and no special facilities are necessary. In addition, by using a microchip having a plurality of microchannels and immobilizing different cells on the basis of a row of micropores, and by introducing different samples by using another microchip having a plurality of microchannels perpendicularly to the row of micropores mentioned above, examinations with different combinations of cells and samples can be conducted on a micropore basis, and examination results corresponding to the number of micropores can be obtained simultaneously. The present invention has been completed based on the above-mentioned findings.

### DISCLOSURE OF THE INVENTION

The present invention relates to: an on-chip bioassay method comprising the steps of: a microfluidic chip for introducing a cell is fixed on the undersurface of a microporous chip composed of a substrate through which a plurality of micropores arranged like a lattice penetrate, and thus forming a plurality of microchannels for introducing a cell between the microporous chip and the microfluidic chip for introducing a cell; a suspended cell is poured into a micropore of the microporous chip through the channel; then a microfluidic chip for introducing a test substance is fixed on the upper surface of the microporous chip such that its plurality of microchannels for introducing a test substance cross over to the plurality of microchannels for introducing a cell, and thus forming a plurality of microchannels for introducing a test substance between the microporous chip and the microfluidic chip for introducing a test substance; a test substance is poured through the channel and brought into contact with the cell in a micropore of the microporous chip; a level of effect of the test substance on the cell is detected in situ after a predetermined time, or at a predetermined time interval (“1”); the on-chip bioassay method according to “1”, wherein a water-resistant breathable sealing membrane is attached to the upper surface of the microporous chip in order to prevent the cell from spilling from the micropore before the suspended cell is poured into the micropore of the microporous chip (“2”); the on-chip bioassay method according to “1” or “2”, wherein the substrate is a silicon substrate (“3”); the on-chip bioassay method according to any one of “1” to “3”, wherein the microfluidic chip is made from polydimethylsiloxane (“4”); the on-chip bioassay method according to any one of “1” to “4”, wherein a cell suspended in a gel is poured into a micropore of the microporous chip and is immobilized in the micropore (“5”); and the on-chip bioassay method according to “5”, wherein a cell suspended in a low melting point agarose gel is used as the cell suspended in a gel (“6”).

The present invention further relates to: the on-chip bioassay method according to any one of “1” to “6”, wherein the microfluidic chip for introducing a cell and the microfluidic chip for introducing a test substance are the same microfluidic chips (“7”); the on-chip bioassay method according to any one of “1” to “7”, wherein temperature is controlled on the basis of a cell row of the micropore of the microporous chip and/or on the basis of a test substance row perpendicular to the cell row (“8”); the on-chip bioassay method according to any one of “1” to “8”, wherein a cell is removed from a micro chip after use and the chip is reused (“9”); the on-chip bioassay method according to any one of “1” to “9”, wherein one or more kinds of cells selected from a group consisting of microbial cells, animal cells and plant cells, are used as a cell (“10”); the on-chip bioassay method according to any one of “1” to “10”, wherein a transformed cell is used as a cell (“11”); the on-chip bioassay method according to any one of “1” to “11”, wherein two or more kinds of test substances are used (“12”); and the on-chip bioassay method according to any one of “1” to “12”, wherein means for detecting a level of effect of a test substance on a cell in situ is a CCD camera with spatial resolution, a photodiode array or a photographic plate, which detects a signal arising from the cell (“13”).

The present invention still further relates to: an on-chip bioassay kit comprising a microporous chip composed of a substrate through which a plurality of micropores arranged like a lattice penetrate for pouring a cell suspended in a gel such as agarose and immobilizing the cell, and two microfluidic chips fixed on each side of the microporous chip and forming a plurality of microchannel groups (“14”); the on-chip bioassay kit according to “14”, further comprising a water-resistant breathable sealing membrane fixed on one side of the microporous chip for preventing a cell from spilling from a micropore (“15”); the on-chip bioassay kit according to “14” or “15”, wherein a temperature control mechanism is provided on the basis of a row and/or a column of micropores on the substrate (“16”); the on-chip bioassay kit according to any one of “14” to “16”, wherein the substrate is a silicon substrate (“17”); the on-chip bioassay kit according to any one of “14” to “17”, wherein the microfluidic chip is made from polydimethylsiloxane (“18”); the on-chip bioassay kit according to any one of “14” to “18”, wherein the micropore is a through-hole of 300 to 900 μm×300 to 900 μm square (“19”); the on-chip bioassay kit according to any one of “14” to “19”, wherein the width of the channel of the microfluidic chip is equal to the length of a side of the micropore (“20”); and the on-chip bioassay kit according to any one of “14” to “20”, wherein the spacing between a plurality of the channels of the microfluidic chip has expanded extremities (“21”).

### BRIEF DESCRIPTION OF DRAWINGS

FIG. 1 is a set of photographs showing micromosaic assay of the present invention.

FIG. 2 is a view showing a microporous chip (A) and a microfluidic chip (B) of the present invention.

FIG. 3 is a set of photographs showing on-chip bioassay in the mutagen detection test of the present invention.

FIG. 4 is a set of photographs showing the results of the mutagen detection test with on-chip bioassay of the present invention.

### BEST MODE OF CARRYING OUT THE INVENTION

There is no particular limitation for the on-chip bioassay method of the present invention as long as it is a method comprising the steps of: a microfluidic chip for introducing a cell is fixed on the undersurface of a microporous chip composed of a substrate through which a plurality of micropores arranged like a lattice penetrate, and thus forming a plurality of microchannels for introducing a cell between the microporous chip and the microfluidic chip for introducing a cell; a suspended cell is poured into a micropore of the microporous chip through the channel; then a microfluidic chip for introducing a test substance is fixed on the upper surface of the microporous chip such that its plurality of microchannels for introducing a test substance cross over (preferably, in a direction almost perpendicular to) the plurality of microchannels for introducing a cell, and thus forming a plurality of microchannels for introducing a test substance between the microporous chip and the microfluidic chip for introducing a test substance; a test substance is poured through the channel and brought into contact with the cell in a micropore of the microporous chip; a level of effect of the test substance on the cell is detected in situ after a predetermined time, or at a predetermined time interval. Here, “to be detected in situ” means that detection is conducted by using a microbioassay chip comprising at least a microporous chip, a microfluidic chip for introducing a cell and a microfluidic chip for introducing a test substance, as it is.

As the microporous chip mentioned above, any microporous chip can be used as long as it is composed of a substrate made from silicon, glass, plastic or the like, through which a plurality of micropores arranged like a lattice of m rows and n columns (m and n may be the same or different, and m×n equals 2 or more) penetrate, and the size of the substrate is not particularly limited, however, substrates of 300 to 1000 μm thick are usually used. The shape of the micropore is not particularly limited and shapes such as square, rectangle, circle, oval, triangle can be exemplified. Among them, square and circle, in particular, square is preferable in that width direction of channels provided on the microfluidic chip and an opening of pore can be conformed. In addition, as to the size of the micropore, 1000 μm or less on a side or in diameter is preferable, and a micropore having a square opening of 300 to 900 μm×300 to 900 μm is specifically exemplified, but its lower limit is not particularly limited. Even in case the sizes of micropores and microchannels are small, it is possible to spread bacterial suspensions and solutions of test substances to the microchannels and micropores by capillary action, and when it is difficult to spread such suspensions and solutions only by capillary action, pressure pumping by high-pressure pumps, or suction by vacuum pumps can be conducted.

Further, a method for penetrating micropores through a substrate by boring is not particularly limited, either. For example, a case of boring of a silicon substrate is described below. A silicon substrate wherein organic matters on its surface has been removed is placed in a thermal oxidation furnace, and the substrate is heated to 1000° C. or higher under nitrogen aeration. Steam is introduced into the furnace to oxidize the silicon substrate so that SiO₂ membrane is formed on its surface. Next, both surfaces of the silicon substrate on which SiO₂ membrane is formed are lipophilized by HMDS, etc., and then photoresist is spin coated and the resulted substrate is prebaked. The prebaked silicon substrate is adhered to a photomask which has been pattern-output to an OHP film with a laser printer, and photolithography is conducted by UV irradiation. Subsequently, etching of the SiO₂ membrane with hydrogen fluoride and anisotropic etching of the silicon substrate with KOH are conducted from both sides of the substrate, and thus micro through-holes can be formed by boring the silicon substrate.

As to the substrate which constitutes a microporous chip, it is preferable to provide a temperature controlling mechanism on the basis of a row and/or a column of micropores. By providing a temperature controlling mechanism on the basis of a row or a column of cells, it becomes possible to simultaneously use cells of plural kinds of microorganisms, etc., that have different optimal growth temperatures. By providing a temperature controlling mechanism on the basis of a row or a column of test substances, it becomes possible to simultaneously detect effects of test substances at different temperatures. A controlling mechanism wherein an optional area on the chip is set at a prescribed temperature by using a printed circuit board as a heater and attaching it to a bioassay chip, is exemplified as such temperature controlling mechanism. On the other hand, for cooling, a controlling mechanism wherein an optional area on the chip is set at a prescribed temperature by attaching Pertier elements in a same manner, is exemplified.

Examples of cells that can be used to the on-chip bioassay of the present invention include microorganism cells, animal cells and plant cells, and more specifically, bacterial procaryotic cells such as Escherichia coli, Streptomyces, Bacillus subtilis, Streptococcus, Staphylococcus; eukaryotic cells such as yeast, Aspergillus; insect cells such as drosophila S2, spodptera Sf9; animal cells such as L cells, CHO cells, COS cells, HeLa cells, C127 cells, BALB/c3T3 cells (including mutant strains deficient in dihydrofolate reductase, thymidine kinase or the like), BHK21 cells, HEK293 cells, Bowes melanoma cells, oocytes, T cells; and plant cells.

In case where the cells are microorganism cells such as Escherichia coli, a cell suspension can be poured into micropores of a microporous chip through a plurality of microchannels for introducing a cell formed between the microporous chip and a microfluidic chip for introducing a cell. Alternatively, the microorganism cells can be used as immobilized microorganisms by pouring cells suspended in a gel hardened by temperature rise or drop into micropores of a microporous chip. Examples of the gel include agar gel, agarose gel, collagen gel, calcium alginate gel, cross-linked dextran gel, synthetic polymer gel. When a synthetic polymer gel is used, polymerization is promoted by using cross-linkers, polymerization promoters, and polymerization initiators together with monomers for a synthetic polymer gel, such as acrylamide and polyvinyl alcohol, and then gelation occurs. Further, in case where the cells are animal cells, suspensions of animal cells attached to collagen gels or supports (carriers) can be used.

In addition, in order to facilitate the detection of a level of effect of a test substance on a cell in situ, transformed cells can be used as the cells. As the transformed cells, a transformant wherein a reporter gene is linked to downstream of a promoter for a candidate gene which expresses by contact with a test substance is exemplified. Such transformants can be constructed by ordinary methods. Specific examples of the reporter gene include: DNAs encoding fluorescent proteins such as GFP (green fluorescent protein); luciferase such as firefly luciferase and bacterial luciferase; enzyme genes such as β-galactosidase. Among them, DNAs encoding fluorescent proteins such as GFP gene are preferable because of easy detection/confirmation. GFP has derivatives with different fluorescent wavelengths such as EGFP (enhanced GFP), EYFP (enhanced yellow fluorescent protein), ECFP (enhanced CYAN fluorescent protein) (blue), DsRed (red), and multiple labeling can be conducted as well. The advantage of GFP is that it can be analyzed easily as live cells, thereby time course observation is easy.

It is possible to prevent the cells from spilling from the micropores by attaching a water-resistant breathable (breathable and impermeable) sealing membrane to the upper surface of the microporous chip before pouring the suspended cells into micropores of the microporous chip. In case where such a water-resistant breathable sealing membrane is used, when a cell suspension is poured into micropores of a microporous chip through a plurality of microchannels for introducing a cell formed between the microporous chip and the microfluidic chip for introducing a cell, no back pressure arises because it is breathable, and water-resistance prevents cell suspensions from leaking from the micropores. This water-resistant breathable sealing membrane will be exfoliate before pouring a test substance through a plurality of microchannels for introducing a test substance formed between the microporous chip and the microfluidic chip for introducing a test substance, however, it is particularly useful when cells in micropores are immobilized, for example, when cells suspended in low melting point agarose gel are used.

The microfluidic chip for introducing a cell is used for forming, in cooperation with the microporous chip, a plurality of microchannels for introducing a cell between the microporous chip and the microfluidic chip for introducing a cell, and poring suspended cells into micropores of the microporous chip through the channels. The width of the channels for introducing a cell formed by fixing the microfluidic chip for introducing a cell on the undersurface of the microporous chip is preferably equal to the length of a side of micropores, and it is preferable that the length makes it possible to spread cell suspensions to all of a plurality of micropores of one row or one column. By pouring different kinds of cell suspensions into respective channels, it becomes possible to introduce different cells into the aligned micropores on a row or a column basis. Cell suspensions or cell-sealing gels in the micropores in the same row or column are connected through the microchannels formed when fixing the microporous chip and the microfluidic chip for introducing a cell together. However, there is no need to exfoliate the microfluidic chip for introducing a cell, or to section gels, etc., analyses can be conducted while the microfluidic chip for introducing a cell and the microporous chip are remain fixed. Further, it is possible to reuse the microfluidic chip for introducing a cell as well as the microporous chip by removing cells from the microchips after use.

The microfluidic chip for introducing a test substance is used for forming, in cooperation with the microporous chip, a plurality of microchannels for introducing a test substance between the microporous chip and the microfluidic chip for introducing a test substance, and bringing a test substance into contact with cells in micropores of the microporous chip through the channels. The width of the channels for introducing a test substance, formed by fixing the microfluidic chip for introducing a test substance on the upper surface of the microporous chip almost perpendicularly to the plurality of microchannels for introducing a cell, is preferably equal to the length of a side of micropores, and it is preferable that the length makes it possible to spread test substances to all of a plurality of micropores in one row or one column. By pouring different kinds of test substances into respective channels, it becomes possible to introduce different test substances into the aligned micropores on a row or a column basis. Test substances brought into contact with cells in the micropores in the same row or column are connected through the microchannels formed when the microporous chip and the microfluidic chip for introducing a test substance are fixed together. However, there is no need to exfoliate the microfluidic chip for introducing a test substance, and analyses can be conducted while the microfluidic chip for introducing a test substance and the microporous chip are remain fixed. Further, it is possible to reuse the microfluidic chip for introducing a test substance as well as the microporous chip by removing test substances from the microchips after use. In addition, examples of the test substances include; various mutagenic substances, endocrine-disrupting chemicals, pharmaceutical candidate substances, heavy metal ions, neurotransmitters, solutions of chemical substances such as cytokines and interleukins, and body fluids such as sera. It is also possible to pour reactants for detection such as enzyme substrates, simultaneously with test substances.

As for these microfluidic chips for introducing a cell and microfluidic chips for introducing a test substance, it is preferable to use the same item with the same shape and made from the same material. The material is not particularly limited as long as it can be fixed on a substrate composing a microporous chip by fixing means, etc., however, materials that can be removably attached to a substrate composing a microporous chip is preferable. For example, in case of a silicon substrate, polydimethyl siloxane (PDMS), which shows high adhesivity to silicon, is particularly preferably exemplified. When PDMS is used, microfluidic chips for introducing a cell and microfluidic chips for introducing a test substance can be constructed by making a mold for PDMS channels, wherein only one side is treated by a method similar to that for constructing the substrate composing a microporous chip mentioned above, and by pouring unpolymerized PDMS and a polymerization initiator into the mold for PDMS channels. PDMS can be advantageously used as a material of microfluidic chips because it has an excellent adhesivity to glass, polymers such as acryl, and PDMS itself, in addition to silicon.

In the on-chip bioassay method of the present invention, one or more kinds of cell suspensions are poured into a microbioassay chip comprising a microporous chip, a microfluidic chip for introducing a cell, and a microfluidic chip for introducing a test substance, and assembled as above, through a plurality of microchannels for introducing a cell formed between the microporous chip and the microfluidic chip for introducing a cell, and subsequently, one or more kinds of test substances are poured through a plurality of microchannels for introducing a test substance formed between the microporous chip and the microfluidic chip for introducing a test substance and the test substances are brought into contact with the cells, a level of effect of the test substances on the cells is detected in situ after a predetermined time, or at a predetermined time interval. For example, signals arising from cells in each micropore can be detected with CCD cameras with spatial resolution, photodiodes such as photodiode arrays, various scanners, photographic plates, etc. By arranging a microfluidic chip for introducing a sample perpendicularly to a microfluidic chip for introducing a cell, tests which use different combinations of cells and samples are employed in each micropore can be conducted simultaneously, and test results corresponding to the number of the micropores can be obtained simultaneously. In addition, the channel design of a microfluidic chip for introducing a cell or a microfluidic chip for introducing a sample can be modified according to the object, and when it is constituted such that the spacing between a plurality of channels on the microfluidic chip has expanded extremities, liquid tubes are connected more easily and injection of cell suspensions and test substances is facilitated.

Next, the on-chip bioassay kit of the present invention is not particularly limited as long as it comprises: a microporous chip composed of a substrate through which a plurality of micropores for pouring suspended cells, arranged like a lattice, penetrate; and two microfluidic chips which are fixed on each side of the microporous chip and forms a plurality of microchannels. As microporous chips and microfluidic chips, the above-mentioned ones can be advantageously used.

The present invention will be more specifically described below with reference to Examples, but the technical scope of the invention will not be limited to these exemplifications.

### EXAMPLE 1

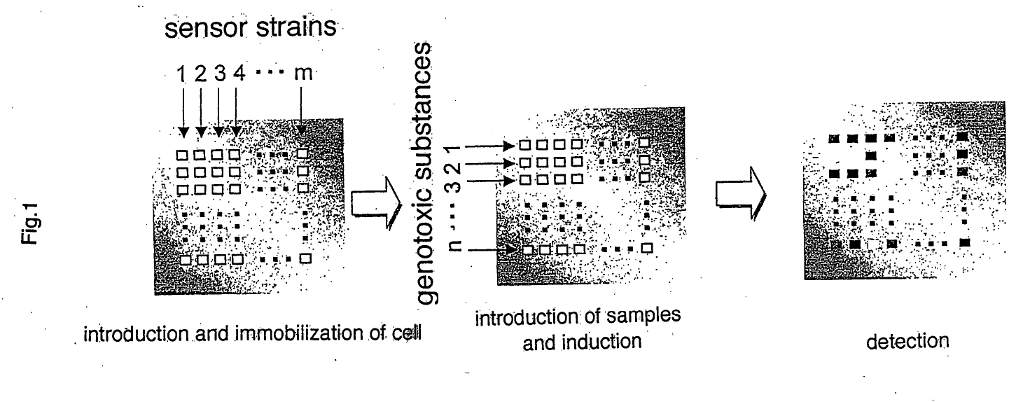

As a specific Example of the present invention, E. coli for mutagenesis assays was immobilized and a micromosaic on-chip bioassay was conducted. As the E. coli for mutagenesis assays, E. coli wherein a gene of bioluminescent firefly luciferase had been incorporated on plasmid instead of SOS gene whose expression is induced by mutagen, was used. Further, the micromosaic assay means, as shown in FIG. 1, an assay which makes it possible to conduct tests of m×n combinations simultaneously by introducing test bacteria into m columns of micropores, and intoroducing samples into n rows of micropores with the use of microfluidic chips. Here, the test was conducted for 5 columns×5 rows. Silicon substrate was used for a microporous chip for immobilizing cells, and PDMS, which shows high adhesivity to silicon and glass, was used for microfluidic chips for introducing E. coli and a sample.

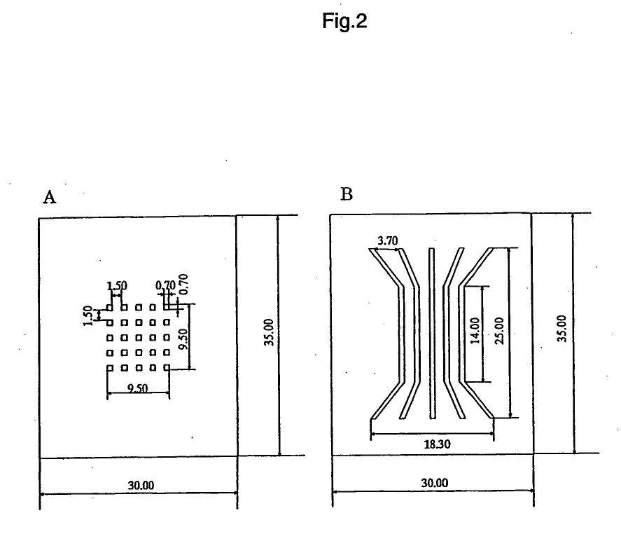

Construction of a microporous chip: a silicon substrate of 3.0×3.5 cm and 625 μm thick was placed in a thermal oxidation furnace, SiO₂ membrane 2 μm thick was formed on its surface. Then, photolithography, etching of the SiO₂ membrane with hydrogen fluoride and anisotropic etching of the silicon substrate with KOH were conducted from both sides of the substrate, and micro through-holes of 700 μm×700 μm square, arranged in 5 rows and 5 columns, 25 holes in total, were formed on the substrate. The spacing between the through-holes was set to 1.5 mm (see FIG. 2A).

### EXAMPLE 2

Construction of microfluidic chips: a silicon substrate was processed in a same manner as mentioned above, and a mold was constructed. PDMS (Dow Corning, SYLGARD184) supplemented with a polymerization initiator was poured into the mold, subjected to reduced-pressure degassing in a glass desiccator for removing mixed air, and left still for 1 hour at 4° C. Subsequently, the resultant was heat-polymerized for 4 hours at 60° C., unmolded, and used as a microfluidic chip. On a microfluidic chip, 5 channels 700 μm wide and 200 μm deep were constructed at 1.5 mm intervals. Two chips, one was for introducing a cell and the other was for introducing a sample, were prepared (see FIG. 2B).

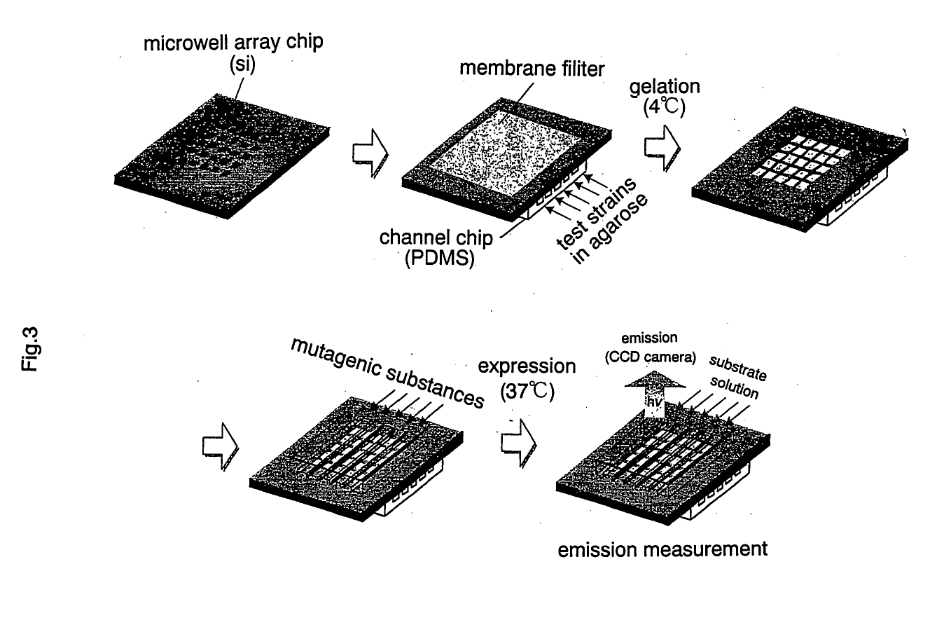

Introduction and immobilization of bacteria for detecting mutagens: E. coli for mutagenesis assays, KT1008/pRSSL, was cultured overnight and added to an LB medium containing 100 μg/mL of ampicillin, in an amount of 1/50 at a volume ratio, and subjected to shaking culture until the absorbance at 37° C. and at a wavelength of 600 nm became about 0.4. Centrifugation was conducted for 5 minutes at 10,000 rpm, and precipitated bacterial cells were suspended in Tris buffered saline. This suspension and 3.0% (w/v) low melting point agarose (Sigma, type VII) were mixed at volume ratio of 1:1. A microfluidic chip was attached to one side of a microporous chip such that the micropores and the channels were overlapped, and test bacteria mixed with agarose were poured thereinto. At that time, a polytetrafluoroethylene membrane filter (Advantec) was attached to the other side of the mocroporous chip to prevent the test bacteria from flowing out. The chip introduced with the test bacteria was left still at 4° C. for 10 minutes, and agarose was gelated. The membrane filter was exfoliated, and the resultant was used as a test bacteria-immobilized chip (see FIG. 3, upper). Here, the same test bacteria were poured into all channels in order to examine reproducibility, etc.

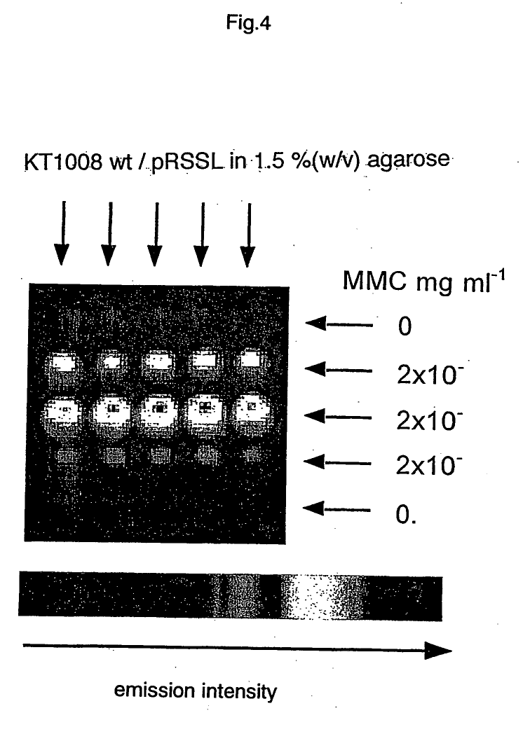

On-chip bioassay: to the side of the test bacteria-immobilized chip, to which a microfluidic chip was not attached, a similar microfluidic chip was attached in a direction perpendicular to that of cell introduction (see FIG. 3, lower). Mitomycin C as a mutagen was poured into the chip, and expression induction was conducted. The optimum condition for the expression induction was 37° C. and 1 hour. Then, in order to conduct bioluminescent detection of the expressed firefly luciferase, a mixed solution of luciferin and adenosine triphosphate was introduced, and photographs of light emitted therefrom were taken with a CCD camera (Hamamatsu Photonics, C-4800) (see FIGS. 1 and 3). Various concentrations of mitomycin C were poured into 5 channels respectively, and as a result, light emission could be observed from each micropore in which test bacteria were immobilized. In addition, emission intensity was dependent on the concentration of mitomycin C, and concentration dependency, similar to that observed in a case conducted in culture solution with the use of test tubes, was observed. Further, little variation was observed for light emission from each micropore in the same column to which the same concentration of mitomycin C was added, and it was confirmed that test bacteria were uniformly immobilized in all micropores (see FIG. 4).

On the basis of the above results, on-chip bioassay of mutagenic substances could be conducted with the use of the microbioassay chip constructed with the microporous chip and the microfluidic chips. By applying this microbioassay chip and on-chip bioassay method to various cells and different types of bioassays, simple tests can be conducted with high throughput. Therefore, it can be said that they are highly useful in various fields which require bioassays.

### INDUSTRIAL APPLICABILITY

It becomes possible to conduct simple tests with high throughput by applying the microbioassay chip and the on-chip bioassay method of the present invention to various cells and different types of bioassays. The bioassay system with this chip-format enables manufacturers to provide chips on which plural kinds of cells are immobilized according to test items, just like current DNA arrays, and enables users to conduct various tests and examinations without preoperations including culture. When combined with a high-sensitive detection method, the development and the microminiaturization of a chip on which cells are high-densely integrated and immobilized can be conducted, and such chip becomes available in various fields including pharmaceuticals, biochemistry and environment.

1. An on-chip bioassay method comprising the steps of: a microfluidic chip for introducing a cell is fixed on the undersurface of a microporous chip composed of a substrate through which a plurality of micropores arranged like a lattice penetrate, and thus forming a plurality of microchannels for introducing a cell between the microporous chip and the microfluidic chip for introducing a cell; a suspended cell is poured into a micropore of the microporous chip through the channel; then a microfluidic chip for introducing a test substance is fixed on the upper surface of the microporous chip such that its plurality of microchannels for introducing a test substance cross over to the plurality of microchannels for introducing a cell, and thus forming a plurality of microchannels for introducing a test substance between the microporous chip and the microfluidic chip for introducing a test substance; a test substance is poured through the channel and brought into contact with the cell in a micropore of the microporous chip; a level of effect of the test substance on the cell is detected in situ after a predetermined time, or at a predetermined time interval.

2. The on-chip bioassay method according to claim 1, wherein a water-resistant breathable sealing membrane is attached to the upper surface of the microporous chip in order to prevent the cell from spilling from the micropore before the suspended cell is poured into the micropore of the microporous chip.

3. The on-chip bioassay method according to claim 1 or 2, wherein the substrate is a silicon substrate.

4. The on-chip bioassay method according to any one of claims 1 to 3, wherein the microfluidic chip is made from polydimethylsiloxane.

5. The on-chip bioassay method according to any one of claims 1 to 4, wherein a cell suspended in a gel is poured into a micropore of the microporous chip and is immobilized in the micropore.

6. The on-chip bioassay method according to claim 5, wherein a cell suspended in a low melting point agarose gel is used as the cell suspended in a gel.

7. The on-chip bioassay method according to any one of claims 1 to 6, wherein the microfluidic chip for introducing a cell and the microfluidic chip for introducing a test substance are the same microfluidic chips.

8. The on-chip bioassay method according to any one of claims 1 to 7, wherein temperature is controlled on the basis of a cell row of the micropore of the microporous chip and/or on the basis of a test substance row perpendicular to the cell row.

9. The on-chip bioassay method according to any one of claims 1 to 8, wherein a cell is removed from a micro chip after use and the chip is reused.

10. The on-chip bioassay method according to any one of claims 1 to 9, wherein one or more kinds of cells selected from a group consisting of microbial cells, animal cells and plant cells, are used as a cell.

11. The on-chip bioassay method according to any one of claims 1 to 10, wherein a transformed cell is used as a cell.

12. The on-chip bioassay method according to any one of claims 1 to 11, wherein two or more kinds of test substances are used.

13. The on-chip bioassay method according to any one of claims 1 to 12, wherein means for detecting a level of effect of a test substance on a cell in situ is a CCD camera with spatial resolution, a photodiode array or a photographic plate, which detects a signal arising from the cell.

14. An on-chip bioassay kit comprising a microporous chip composed of a substrate through which a plurality of micropores arranged like a lattice penetrate for pouring a cell suspended in a gel such as agarose and immobilizing the cell, and two microfluidic chips fixed on each side of the microporous chip and forming a plurality of microchannel groups.

15. The on-chip bioassay kit according to claim 14, further comprising a water-resistant breathable sealing membrane fixed on one side of the microporous chip for preventing a cell from spilling from a micropore.

16. The on-chip bioassay kit according to claim 14 or 15, wherein a temperature control mechanism is provided on the basis of a row and/or a column of micropores on the substrate.

17. The on-chip bioassay kit according to any one of claims 14 to 16, wherein the substrate is a silicon substrate.

18. The on-chip bioassay kit according to any one of claims 14 to 17, wherein the microfluidic chip is made from polydimethylsiloxane.

19. The on-chip bioassay kit according to any one of claims 14 to 18, wherein the micropore is a through-hole of 300 to 900 μm×300 to 900 μm square.

20. The on-chip bioassay kit according to any one of claims 14 to 19, wherein the width of the channel of the microfluidic chip is equal to the length of a side of the micropore.

21. The on-chip bioassay kit according to any one of claims 14 to 20, wherein the spacing between a plurality of the channels of the microfluidic chip has expanded extremities.
